(19) United States
(12) Patent Application Publication
(10) Pub. No.: US 2004/0138597 A1
(43) Pub. Date: Jul. 15, 2004

(54) Dorsal blocking hand orthosis

(75) Inventor: Harold T. Varn, Lawrenceville, GA (US)

Correspondence Address: ZARLEY LAW FIRM P.L.C., CAPITAL SQUARE, 400 LOCUST, SUITE 200, DES MOINES, IA, 50309-2350, US

(73) Assignee: Restorative Care of America, St. Petersburg, FL (US)

(21) Appl. No.: 10/341,038

(22) Filed: Jan. 13, 2003

(51) Int. Cl. A61F 5/00

(52) U.S. Cl. 602/21; 602/20

(57) ABSTRACT

A dorsal blocking hand orthosis has a rigid hand splint member which has a forearm portion attached to a finger portion, which are adapted to receive a patient's hand so that a forearm portion and a splint member rest on the dorsal portion of the patient's wrist and forearm, and the finger portion receives the patient's fingers.

### BACKGROUND OF THE INVENTION

Currently dorsal blocking hand splints must be made from scratch by the therapist using kits typically containing strapping and precut thermoplastic material. Unfortunately, this customization requires more of the therapist's time and expensive materials, leading to a high overall cost. Current dorsal blocking orthosis do not adequately prevent unwanted finger and wrist extension, nor adequately aid in finger flexion exercises.

It is therefore a principle object of this invention to provide a dorsal blocking hand orthosis which offers prevention of unwanted extension of the fingers and wrist.

A further object of this invention is to provide a dorsal blocking hand orthosis which aids in finger flexion exercise.

A still further object of this invention is to assist in restoring functional motion to the fingers.

A still further object of this invention is to prevent inadvertent removal of the orthosis.

A still further object of this invention is to provide a dorsal blocking hand orthosis having a liner that is not affixed to the supporting splint by structural features of the splint itself.

A still further object of this invention is to provide a dorsal blocking hand orthosis which will provide a time- and cost-saving alternative to therapists.

These and other objects will be apparent to those skilled in the art.

### BRIEF SUMMARY OF THE INVENTION

The foregoing objects may be achieved by a dorsal blocking hand orthosis. The invention comprises a substantially rigid hand splint member which has a forearm portion attached to a finger portion. The dorsal blocking hand orthosis of this invention is adapted to receive a patient's hand such that the forearm portion and splint portions rest on the dorsal portion of the patient's wrist and forearm, and the finger portion receives the patient's fingers.

A liner is located on the inner surface of the substantially rigid splint member. Strap elements secure to the patient. The straps are redundantly secured. A buckle connected to one of the straps aids in finger flexion exercises.

### BRIEF DESCRIPTION OF THE DRAWINGS AND PHOTOS

FIG. 1 is a perspective view of the dorsal blocking hand orthosis of this invention, showing primarily the upper surface thereof and including a patient's hand in the general position that is held by the device of this invention.

FIG. 2 is a perspective view of the dorsal blocking hand orthosis of this invention, showing primarily the lower surface thereof.

FIG. 3 is a view similar to FIG. 1 but has the liner, attachment straps, and patient's hand removed therefrom.

FIG. 4 is a view similar to FIG. 2 but shows the liner, attachment straps, and patient's hand removed therefrom.

FIG. 5 is a plan view of the detached liner.

FIG. 6 is a view similar to that of FIG. 5 but is shown in a smaller scale so as to show all attachment straps, as shown in FIG. 2.

FIG. 7 is a left side elevational view, assuming dorsal blocking hand orthosis depicted is for the right hand.

FIG. 8 is a right side elevational view of the dorsal blocking hand orthosis shown in FIG. 7.

### DESCRIPTION OF THE PREFERRED EMBODIMENTS

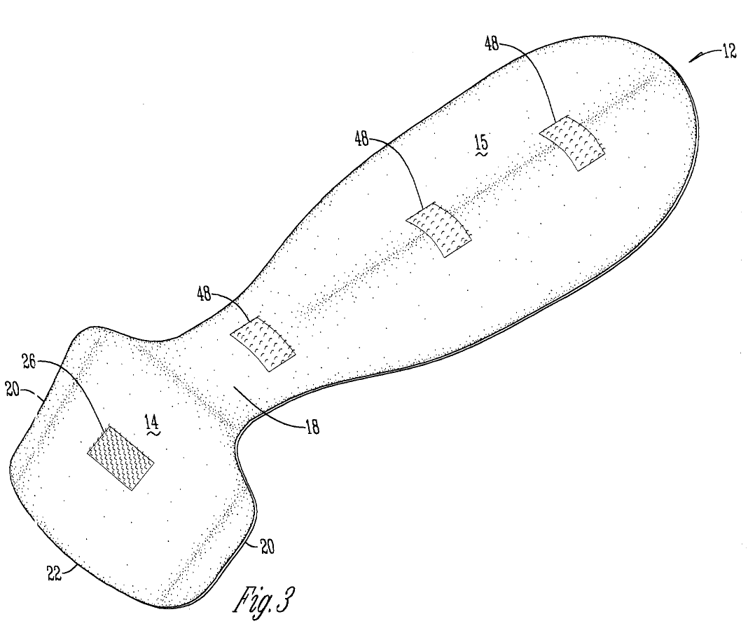

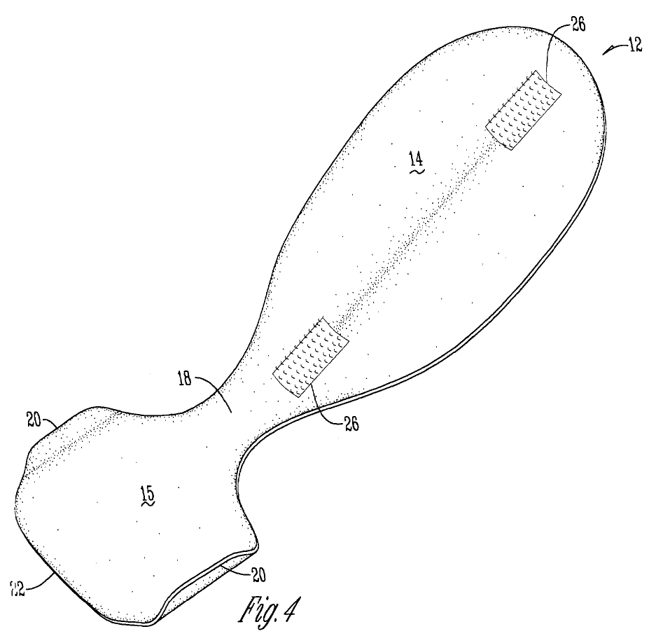

The dorsal blocking orthosis 10 has a substantially rigid splint 12 best shown in FIGS. 3 and 4. The splint 12 has an inner surface 14 and an outer surface 15. With reference to FIG. 3, the splint 12 includes a forearm portion 18, two lateral supports 20; and a finger portion 22. For purposes herein, the forearm portion 18 is deemed to include what also might be called a wrist portion.

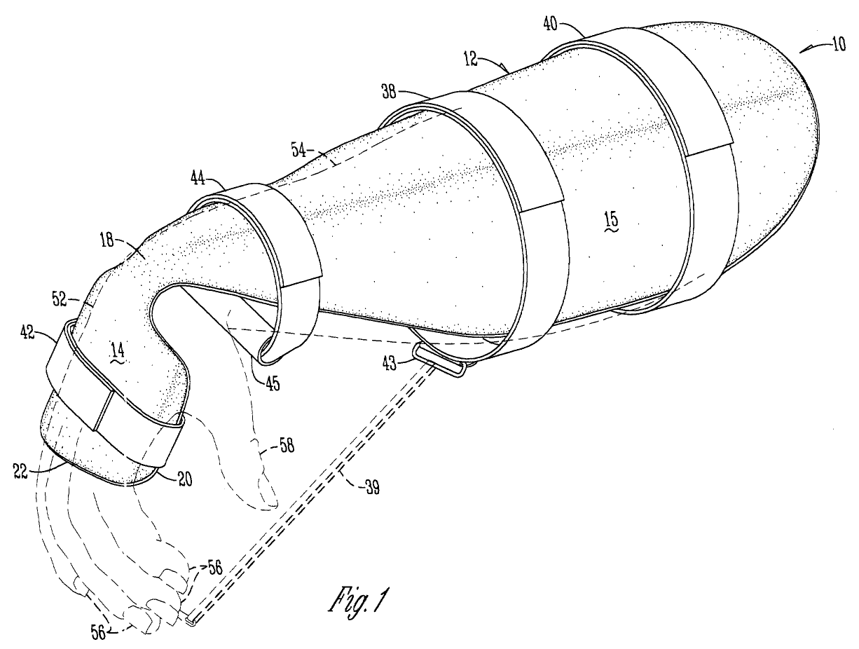

As will be described hereafter, a plurality of Velcro® fasteners are used in this invention which include hook patches which can be detachably connected to material having a plurality of loops. A plurality of hook patches 26 are permanently secured to the splint 12 on the inner surface 14 to secure a flexible, resilient, one-piece liner 28 in position on the inner surface 14 of the splint 12. Referring to FIG. 1, additional hook patches 48 are permanently secured to the splint 12 on the outer surface 15 thereof to secure the wrist straps 38, 40 to the splint 12.

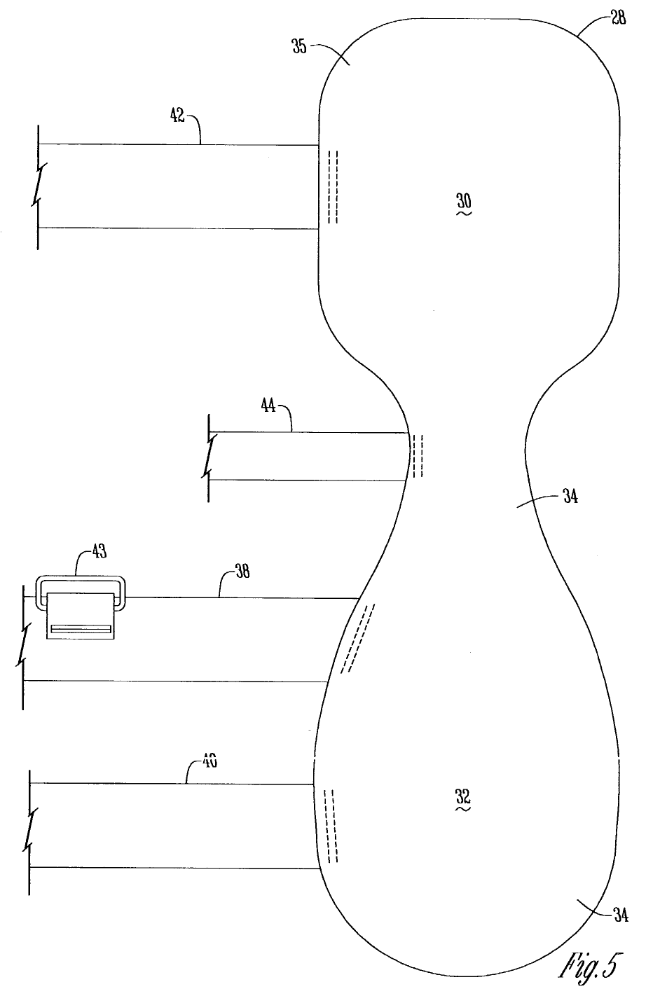

With reference to FIG. 5, the liner 28 has an inner side 30 and an outer side 32. Said liner 28 has a wrist portion 34, and a finger portion 35.

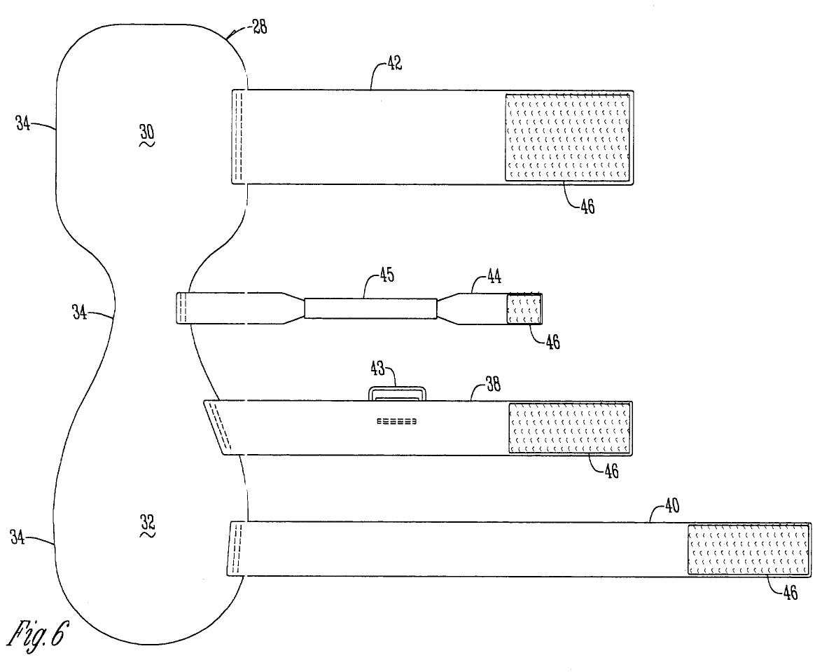

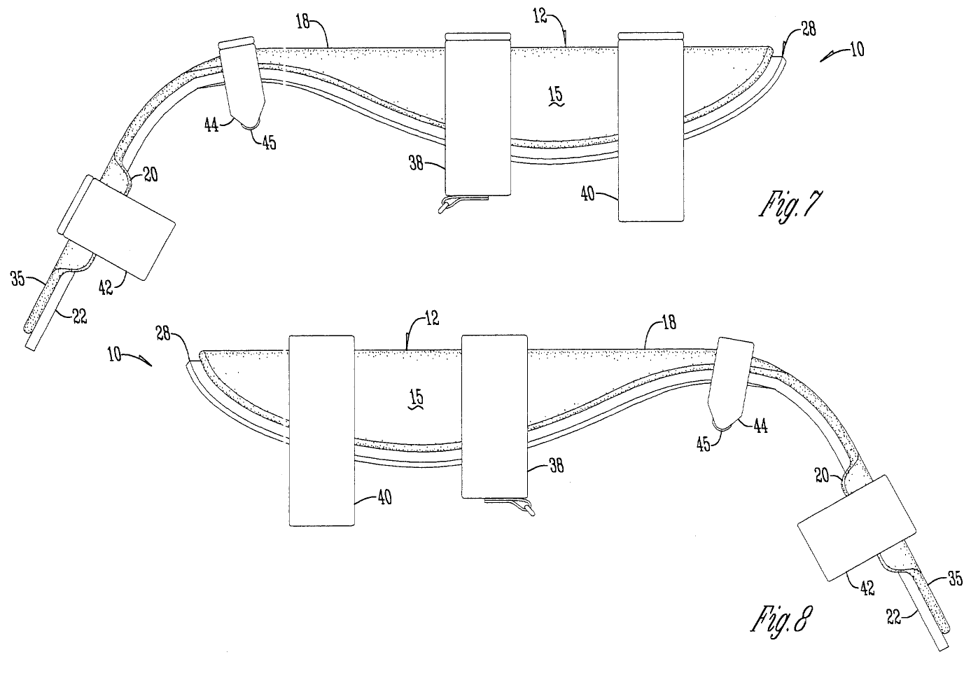

With reference to FIG. 6, elongated wrist straps 38, 40, 42, and 44 are secured to the liner's inner side 30 by one of their ends. Wrist straps 38 and 40 are attached to the forearm portion of the liner 34. The finger strap 42 is attached to the finger portion of the liner 35. The strap 44 is attached to liner 36 approximately where a patient's wrist flexes. All straps 38, 40, 42, and 44 have hook patches 46 attached to the end opposite of the point of attachment to the liner 28. Each of the straps is comprised of loop material which is adapted to be detachably secured to the hook patches 46, 48. Strap 44 is inserted through a hollow cylinder 45. The hollow cylinder provides comfort for the patient. Strap 38, which is generally positioned near the volar of the wrist includes a buckle 43. A flexible member 47 such as a rubberband is attached to the buckle at one end and is hooked to the patient's finger to provide resistance for finger flexion exercises.

The liner 28 is comprised of a full material, which along with the material of the straps as described heretofore, absorbs perspiration away from the patient's skin, thus promoting good skin integrity.

With reference to FIG. 1, the patient's forearm, wrist, and hand are depicted. The numeral 54 designates the patient's hand. Numeral 56 designates the patient's fingers. Numeral 58 designates the patient's thumb. Numeral 60 designates the patient's knuckles. In operation, the patient's hand 52 is inserted into the dorsal blocking orthosis 10. The patient's fingers 56 rest on the finger portion of the liner 35. The patient's wrist and forearm engage the forearm portion of the liner 34.

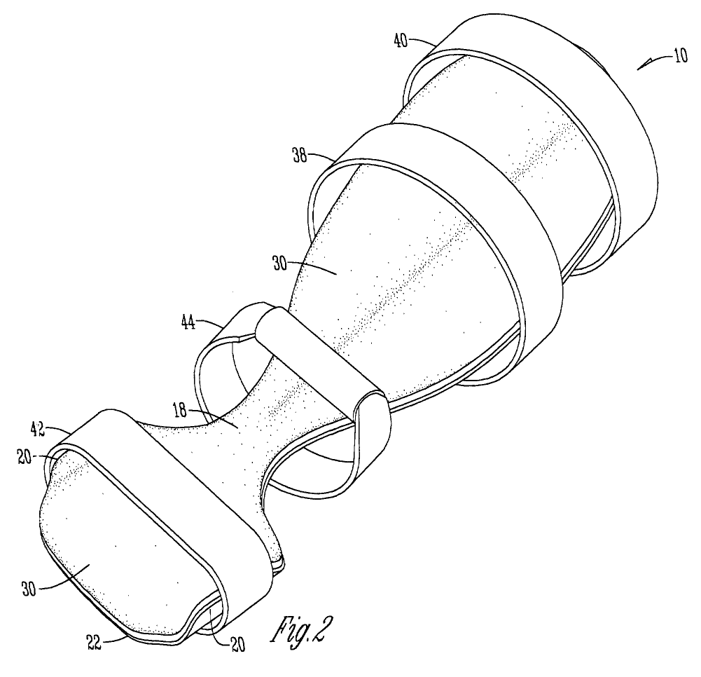

The wrist straps 38, 40, and 44 can then be extended around the wrist and forearm of the patient and secured upon the outer surface 15 of the substantially rigid plastic splint 12 with the hook patches 48 attached thereto. The wrist straps 38, 40, and 44 can then be extended around the wrist and forearm of the patient and secured upon the outer surface 15 of the substantially rigid plastic splint 12 with the hook patches 48 attached thereto. The wrist straps 38, 40, and 44 can further be secured to themselves as is best shown in FIG. 2. Similarly, the finger strap 42 can be extended around the fingers of the patient 56 engaging the hook patch 48 and secured upon itself by hook patch 46.

When it is desired to change the liner 28, the entire liner can be detached from the splint 12 by unfastening the straps 38, 40, 42, and 44 in conventional manner to place the liner in the condition shown in FIG. 5. Thus, the liner can easily be removed in one piece without having, for example, to unthread straps through holes or slots in the splint.

It is therefore seen that this device will achieve all of its stated objectives.

What is claimed is:

1. A dorsal blocking orthosis for a patient, comprising:
a substantially rigid dorsal contoured splint having an outer forearm portion adapted to fit on and receive the dorsal portion of the wrist and forearm of the patient and a finger portion spaced forwardly from said wrist portion to receive the fingers of the patient,
a means to secure the dorsal blocking orthosis to the patient and prevents the patient's finger and wrist extension; and
the splint having an inner and outer surface.

2. The device of claim 1 wherein the inner surface is covered by a resilient liner.

3. The device of claim 2 wherein the liner is of one-piece construction.

4. The device of claim 2 wherein the splint and the liner are of one-piece construction.

5. The device of claim 2 wherein at least one patch is secured to the inner side of the splint to detachably cling to the liner.

6. The device of claim 1 wherein the means to secure and prevent extension comprises strap elements adapted to extend around the splint and the patient's forearm, wrist, and fingers.

7. The device of claim 6 wherein patches are secured to the outer surface of the splint to detachably cling to the straps.

8. The device of claim 6 wherein at least one of the straps is received within and extends through a hollow cylindrical body.

9. The device of claim 6 further comprising a means for aiding finger flexion.

10. The device of claim 9 wherein the means for aiding finger flexion comprises a buckle attached to a strap, the buckle connected to the patient's finger by a flexible member.

11. A dorsal blocking orthosis for a patient, comprising:
a substantially rigid dorsal contoured splint having an outer forearm portion adapted to fit on and receive the dorsal portion of the wrist and forearm of the patient and a finger portion spaced forwardly from said wrist portion to receive the fingers of the patient,
a means to secure the dorsal blocking orthosis to the patient and prevents the patient's finger and wrist extension;
the splint having an inner and outer surface; and
a means for aiding finger flexion.

12. The device of claim 11 wherein the inner surface is covered by a resilient liner.

13. The device of claim 11 wherein the liner is of one-piece construction.

14. The device of claim 11 wherein the splint and the liner are of one-piece construction.

15. The device of claim 11 wherein at least one patch is secured to the inner side of the splint to detachably cling to the liner.

16. The device of claim 11 wherein the means to secure and prevent extension comprises strap elements adapted to extend around the splint and the patient's forearm, wrist, and fingers.

17. The device of claim 16 wherein patches are secured to the outer surface of the splint to detachably cling to the straps.

18. The device of claim 16 wherein at least one of the straps is received within and extends through a hollow cylindrical body.

19. The device of claim 16 wherein the means for aiding finger flexion comprises a buckle attached to a strap, the buckle connected to the patient's finger by a flexible member.

20. A dorsal blocking orthosis for a patient, comprising:
a substantially rigid dorsal contoured splint having an outer forearm portion adapted to fit on and receive the wrist and forearm of the patient and a finger portion spaced forwardly from said wrist portion to receive the fingers of the patient,
the splint having an inner and outer surface;
the inner surface covered by a resilient liner having strap elements adapted to extend around the outer surface of the splint and the patient's forearm, wrist, and fingers; and
a buckle attached to at least one of the straps.
